(19) United States
(12) Patent Application Publication
(10) Pub. No.: US 2019/0082960 A1
(43) Pub. Date: Mar. 21, 2019

(54) SYSTEMS AND METHODS FOR MONITORING BONE FUSION

(71) Applicant: Deborah Munro, Portland, OR (US)

(72) Inventor: Deborah Munro, Portland, OR (US)

(21) Appl. No.: 15/706,372

(22) Filed: Sep. 15, 2017

(51) Int. Cl. A61B 5/00

(52) U.S. Cl. CPC A61B 5/0031; A61B 2090/064; A61B 2562/0261; A61B 5/4566

(57) ABSTRACT

Systems and methods utilize a strain sensor to monitor the status of bone fusion. The systems include a sensor system that measures an initial strain in a medical device implanted in or disposed on a bony structure to obtain an initial strain value, and measures a subsequent strain in the medical device to obtain a subsequent strain value. The systems also include a reader or a processor that calculates a magnitude of the difference between the subsequent strain value and the initial strain value or a magnitude of the difference between a current strain value and a previous strain value. If the magnitude of the difference is greater than the predetermined difference value, the sensor system measures multiple strain values, and the processor calculates an average slope based on the multiple measured strain values, and, if the average slope is less than the predetermined slope, generates a notification that bone fusion in a bony structure has occurred.

### BACKGROUND

### 1. Technical Field

This disclosure generally pertains to performing bone fusion procedures, and more particularly to systems and methods for tracking the status and progress of bone fusion using strain measurements.

### 2. Discussion of Related Art

One of the ongoing challenges facing orthopedic surgeons is determining solid bone fusion after spinal fusion surgery. During spinal fusion surgery, the surgeon places morselized bone and/or other bone growth factors in or around the spinal hardware or device. The morselized bone triggers a fracture healing response, causing stem cells and other repair cells to begin growing new bone at the fusion site. This new bone is cartilaginous and radiographically translucent. Thus, although the new bone has significant mechanical strength after about two or three months, surgeons cannot see the fusion using conventional radiographic or other imaging techniques until after at least four months.

Bone fusion becomes visible after the new bone has reached full size and the body begins to infuse the cartilaginous mass with calcium and apatite to convert the new bone into bone tissue. In addition, the metal of the spinal implants has a large radiographic artifact, which makes it difficult to see bony details of the developing fusion. Also, the spinal implants themselves are bulky and obscure the fusion site. Furthermore, patients can only be positioned either on their back or on their side, and this orientation may not be the appropriate orientation needed to view the fusion. And even after seven to nine months, the accuracy rate for predicting solid bone fusion may only be 70 percent.

### SUMMARY

In aspects, the present disclosure features a method of monitoring spinal fusion that addresses the potential challenges with conventional techniques for monitoring spinal fusion. The method includes measuring an initial strain in a medical device implanted in a bony structure to obtain an initial or baseline strain value and measuring a subsequent strain in the spinal device to obtain a subsequent strain value. The method further includes calculating a difference between the subsequent strain value and the initial strain value and determining whether the difference is greater than a predetermined difference value. The predetermined difference value may be determined based on prior testing or analysis of a similar bony structure that has been subjected to a fusion process. The predetermined difference value may also be based on the patient's medical data including particular characteristics of all or a portion of the patient's skeletal system.

The method further includes, in response to determining that the difference is greater than the predetermined difference value, measuring multiple strain values, calculating an average slope based on the multiple measured strain values, and determining whether the average slope is less than a predetermined slope. The method further includes, in response to determining that the average slope is less than the predetermined slope, generating a notification or message that bone fusion in the bony structure has occurred.

In some aspects, the method includes determining the rate of change in the difference between adjacent measured strain values and determining whether the rate of change in the difference between adjacent measured strain values does not exceed a predetermined rate of change. If the rate of change in the difference between adjacent measured strain values does not exceed a predetermined rate of change, the method may include generating a notification that the bone fusion process is not progressing properly.

In some aspects, the method further includes, in response to determining that the average slope is greater than the predetermined slope, generating a notification or a message that bone fusion in the bony structure has not occurred. In other aspects, the method further includes determining whether a predetermined period has elapsed and, in response to determining that the difference is less than the predetermined difference value, the average slope is greater than the predetermined slope, and the predetermined period has elapsed, generating a notification or a message that a non-union or pseudo-union in the bony structure has occurred.

In aspects, the method further includes transmitting a wireless signal including the initial strain value and the subsequent strain value from a strain sensor disposed in the medical device to a reader device.

In aspects, each of the strain values is measured at a predetermined interval. The predetermined interval may be one day or one week.

In aspects, the method further includes measuring strain at multiple positions on the spinal device at different times to obtain multiple strain values, calculating multiple differences between multiple initial strain values of the multiple measured strain values and multiple subsequent strain values of the multiple measured strain values, and determining whether each of the differences is greater than a respective predetermined difference value of multiple predetermined difference values. The method further includes, in response to determining that each of the differences is greater than the respective predetermined difference values, measuring strain at multiple positions of the spinal device at different times to obtain multiple sets of strain values, calculating an average slope for each of the multiple sets of strain values based on the multiple sets of strain values, and determining whether the average slope for each of the multiple sets of strain values is less than a predetermined slope. The method further includes, in response to determining that the average slope for each of the multiple sets of strain values is less than the predetermined slope, generating a notification or message that bone fusion in the bony structure has occurred.

In aspects, the medical device may be a spinal device or a device configured for treating spinal structures, foot structures, leg structures, or arm structures, e.g., a long bone device or a device configured for treating arm bones or clavicles. In aspects, the spinal device is a spinal rod or a spinal plate.

In aspects, the method further includes transmitting a wireless interrogation signal from a wireless reader to a strain sensor embedded in the medical device, and, in response to receiving the wireless interrogation signal at the strain sensor, powering on the strain sensor, measuring the current strain in the spinal device, and transmitting strain information based on the measured strain from the strain sensor to the wireless reader. In aspects, the strain sensor is powered by the wireless interrogation signal.

In aspects, the method further includes transmitting the strain information from the wireless reader to a computing device, and performing the calculating and determining steps by the computing device.

In aspects, the bony structures are spinal structures, foot structures, leg structures, or arm structures.

In aspects, the multiple measured strain values include the subsequent strain value.

In aspects, the present disclosure features a system for performing spinal fusion. The system includes a spinal device, a strain sensor mechanically coupled to the spinal device, and a computing device in communication with the spinal device. The strain sensor periodically measures strain in the spinal device deployed at the spine. The computing device is in communication with the spinal device to acquire an initial strain value from the spinal device and acquire a subsequent strain value from the spinal device. The computing device also calculates a difference between the subsequent strain value and the initial strain value, determines whether the difference is greater than a predetermined difference value, and if it is determined that the difference is greater than the predetermined difference value, measures multiple strain values, calculates an average slope based on the multiple measured strain values, and determines whether the average slope is less than a predetermined slope. Also, if the computing device determines that the average slope is less than the predetermined slope, the computing device determines that spinal fusion has occurred.

In aspects of the system, the reader device further causes the strain sensor to measure an initial strain in the spinal device and causes the strain sensor to measure a subsequent strain in the spinal device.

In aspects of the system, the computing device is a reader device including a first wireless transceiver that wirelessly acquires strain values from a second wireless transceiver disposed in the spinal device and coupled to the strain sensor.

In aspects, the spinal device is a screw, a spinal rod, a cervical plate, a spinal plate, a spinal implant, or an artificial disc.

In aspects, the multiple strain values are measured at a predetermined interval. In aspects, the predetermined interval is one day or one week.

### BRIEF DESCRIPTION OF THE DRAWINGS

Various aspects of the present disclosure are described below with reference to the drawings, wherein:

FIG. 1 is a block diagram illustrating a system architecture for monitoring the status of spinal fusion in accordance with some embodiments;

FIG. 2 is a block diagram illustrating a sensor system employed in the spinal device of the system architecture of FIG. 1;

FIG. 3 is a block diagram illustrating a reader of the system architecture of FIG. 1;

FIG. 4 is a perspective view of a spinal plate on which a sensor system is disposed in accordance with some embodiments;

FIG. 5 is a graphical diagram illustrating measurements of strain in a spinal device over time after a spinal fusion procedure in accordance with embodiments of the present disclosure;

FIG. 6 is a flow diagram illustrating an example process for monitoring the status of bone fusion in accordance with some embodiments of the present disclosure;

FIG. 7 is a flow diagram illustrating an example process for monitoring the status of bone fusion in accordance with an embodiments of the present disclosure;

FIG. 8 is a flow diagram illustrating an example process for monitoring the status of bone fusion in accordance with other embodiments of the present disclosure; and

FIG. 9 is a flow diagram illustrating an example process for monitoring the status of bone fusion in accordance with other embodiments of the present disclosure.

### DETAILED DESCRIPTION

Embodiments of the present disclosure are now described in detail with reference to the drawings in which like reference numerals designate identical or corresponding elements in each of the several views. As used herein, the term “clinician” refers to a doctor, a nurse, or any other care provider and may include support personnel. Throughout this description, the phrase “in embodiments” and variations on this phrase generally is understood to mean that the particular feature, structure, system, or method being described includes at least one iteration of the disclosed technology. Such phrase should not be read or interpreted to mean that the particular feature, structure, system, or method described is either the best or the only way in which the embodiment can be implemented. Rather, such a phrase should be read to mean an example of a way in which the described technology could be implemented, but need not be the only way to do so.

A study by Kanayama in 1997 showed that strain can be used to measure the stiffness of a spinal fusion. In the study, sheep spines were harvested at various time intervals of four, eight, twelve, and sixteen weeks. After harvesting the sheep spines, one of the spinal rods was replaced with a rod instrumented with strain gauges. The graphs of the data obtained from the strain gauges showed the basic behavior of a developing spinal fusion at discreet time points. After an initial settling-in period with swelling, the strain in the rod was at a maximum. As the cartilaginous woven bone grew around the spinal hardware, it stiffened the spinal construct and was load sharing with the spinal rod. This caused the strain in the rod to decrease. After eight weeks, the observed strain plateaued at a minimal value and remained relatively constant, indicating bone fusion had occurred, even though it could not be seen with traditional radiographic techniques.

The systems and methods of the present disclosure significantly reduce the amount of time required to determine whether bone fusion has occurred. In embodiments, the techniques of the present disclosure are independent of radiographs or other types of medical images. In embodiments, the technique gathers, interpolates, and condenses large amounts of data transmitted from the sensor system into simple results (e.g., a single yes/no answer, an indication that a threshold has been achieved, or an indication that the output is abnormal (for example, a screw is broken)), and may not require interpretation of a graph. The results may be conclusive and provide evidence-based diagnostics.

The systems and methods of the present disclosure include a sensor system that may be embedded in a spinal plate or a spinal rod. The sensor system may transmit measurement data wirelessly to an external reader. The external reader can interface with a computer for long term storage of a patient's data. The reader can also work independently with its own internal storage. The methods of the present disclosure may be embodied in a software algorithm that can run in the external reader or that can be run on the computer. The external reader may be handheld, attached to the computer, or attached to the patient. The external reader may be in close enough proximity to the patient to allow for the reliable transmission of the wireless signal. As patient privacy is critical, the signal transmission is secure and not readable by other wireless devices other than the external reader. For example, the sensor signals may be encrypted by the sensor system prior to being transmitted to or read by the external reader. In some embodiments, the reader provides the surgeon with a rapid yes/no answer on the status of the developing fusion that the surgeon can compare to the radiographs at the surgeon's discretion.

The methods of the present disclosure use two independent criteria to assess bone fusion: (1) the slope of the strain data curve and (2) the step change in the magnitude of the strain. Bone fusion cannot be assessed without the two independent criteria, as a flat slope can mean several things: solid fusion, delayed or pseudo-union, or non-union. The methods of the present disclosure repeatedly measure the average of the slope across two or more consecutive strain values obtained during a given period. In embodiments, the prior strain values are stored in the external reader or on the computer in the patient's file and are used to calculate the slope.

Thus, a complete history of the developing fusion is known for the patient, and trends and changes can be seen not only immediately after surgery but years later. For instance, if the patient begins to experience pain or a change in range of motion, their current levels of strain can be compared to their historical data. Spinal fusion revision is sometimes necessary when a pedicle screw breaks, a spinal rod becomes loose, or the endplate loading on the vertebrae adjacent to the fusion changes. Other times the surgical outcome is poor and the bony structures may not fuse (non-union) or may develop a cartilaginous pseudo-union that does not convert into bone. Using empirical data from biomechanical testing, these conditions may be characterized and stored in the software algorithm to provide likely causes of the patient's complaint or condition. This may provide the surgeon with additional diagnostic data to determine next steps for their patient.

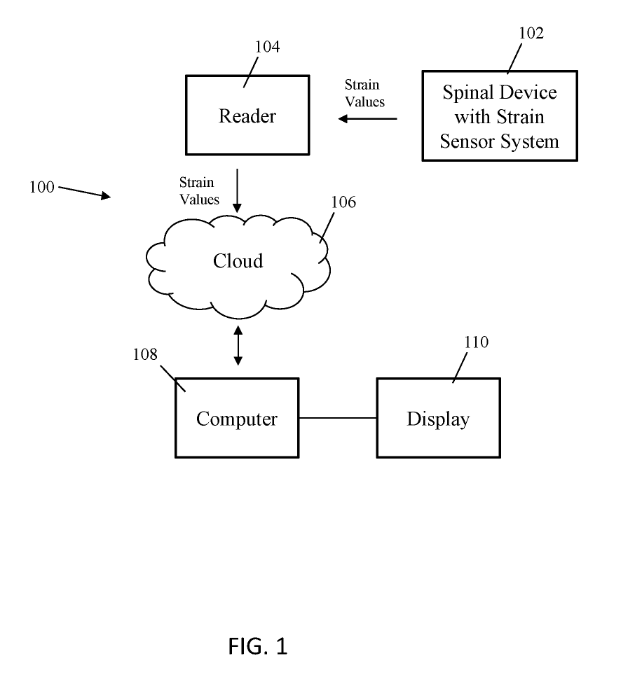

FIG. 1 is a block diagram illustrating a system architecture 100 for monitoring the status of spinal fusion in accordance with some embodiments. A spinal device 102 is implanted or deployed in the spine. The spinal device 102 incorporates a strain sensor system that provides sensed strain values to a reader 104. The spinal device 102 may be a screw, a spinal rod, a cervical plate, a spinal plate, a spinal implant, an artificial disc, or a growing rod. In some embodiments, the reader 104 may provide a power signal to the strain sensor system so that it can sense the strain values at that time. In other embodiments, the strain sensor system may include a storage device so that the strain sensor system can sense the strain values at regular intervals and store them internally in the storage device. Then, after a predetermined period, the reader 104 reads the strain values from the strain sensor system's internal memory.

After obtaining a sufficient number of strain values, the reader 104 may analyze the strain values to determine the status of the spinal fusion or the reader 103 may transmit the strain values to a computer 108 via a cloud computing system 106. For example, the strain values are stored in the cloud computing system 106 and the stored strain values are acquired from the cloud computing system 106. The computer 108 may analyze the strain values to determine the status of the spinal fusion and the status may be displayed on display 110. Alternatively, the reader 104 may perform a portion of the analysis and the computer 108 may perform the remaining portion of the analysis. As yet another alternative, the reader 104 may perform all or a large portion of the analysis and transmit the result to the computer 108, which may then perform a process to interpret the analysis result and display the interpretation.

In embodiments, the reader 104 may be a wearable device that continuously and regularly reads sensor data from the strain sensor system. The reader 104 may also continuously calculate a slope based on the read sensor data and determine whether the magnitude of the calculated slope is greater than a predetermined slope indicating that bone fusion has started or is properly progressing. If the reader 104 determines that the magnitude of the calculated slope is not greater than the predetermined slope, then the reader 104 may generate an alarm, e.g., an audible alarm, and/or may transmit a message to a clinician indicating that bone fusion has not started or is not progressing properly. In embodiments, the reader 104 may be integrated into a multifunction electronic device such as a smartphone. For example, the reader 104 may be implemented by software running on a smartphone that controls a communications interface and/or hardware (e.g., an antenna) to read sensor data from the strain sensor system.

In embodiments, the spinal device includes a communications device configured to communicate with the cloud computing system 106 and configured to transmit and store strain values in memory or a database of the cloud computing system 106. The database may be a database that stores electronic medical records. Strain values may be stored in the electronic records of the patient in which the spinal device has been implanted. In some embodiments, the computing device or computer 108 acquires strain values from the cloud computing system 106. In other embodiments, the computing device or computer 108 is at least a portion of the cloud computing system 106.

In embodiments, the reader 104, which may be a computing device, is a portable electronic device, a smartphone executing an application, or a personal computer executing a software application configured to acquire strain values from the strain sensor of the spinal device.

In some embodiments, the reader 104 acquires strain values from the strain sensor of the spinal device by transmitting an electromagnetic signal to the strain sensor to cause the strain sensor to sense and transmit a strain value to the reader 104. In other embodiments, the reader 104 acquires strain values from the strain sensor of the spinal device by generating an electrical field and inducing a current in a coil of the strain sensor to cause the strain sensor to sense and transmit an electromagnetic signal including strain value information to the reader 104.

In embodiments, the spinal device includes memory for storing strain values, and the reader 104 acquires the strain values from the memory when the computing device comes in close proximity to the strain sensor of the spinal device.

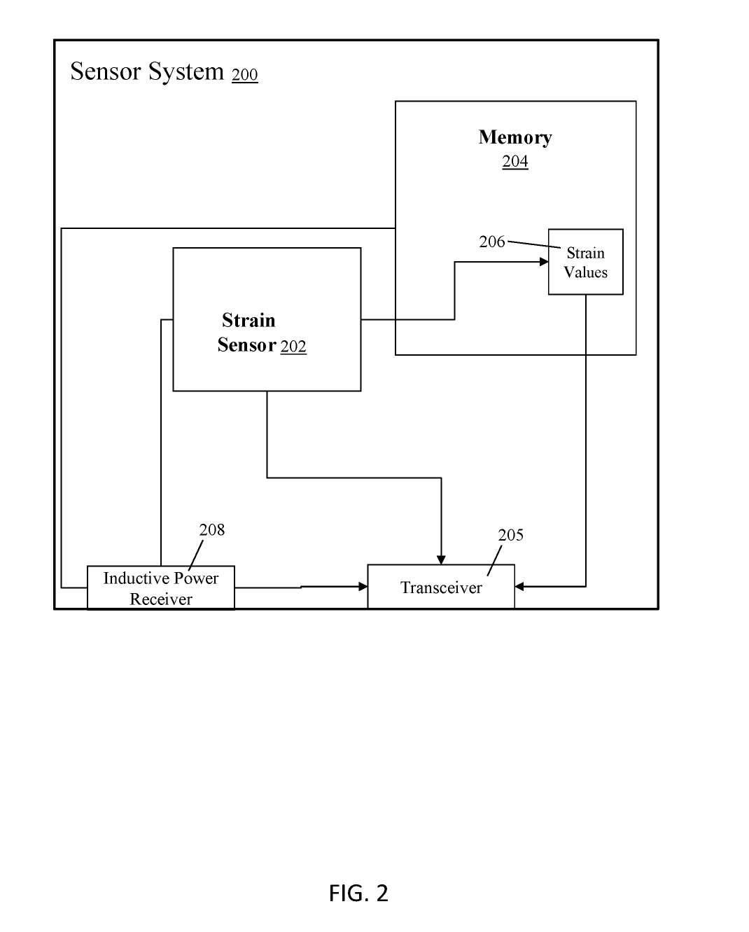

FIG. 2 is a block diagram illustrating a strain sensor system employed in the spinal device of the system architecture 100 of FIG. 1. The sensor system 200 includes a strain sensor 202, a non-volatile memory 204 coupled to the strain sensor 202, and a transceiver coupled to the strain sensor 202 and the non-volatile memory 204. The sensor system 200 also includes an inductive power receiver 208 that includes an inductive element, e.g., a coil, in which a current is induced by an inductive power transmitter in a device external from the sensor system 200.

The strain sensor 202 may be any of the strain sensors disclosed in commonly-owned U.S. Pat. No. 9,510,785, the entire contents of which are incorporated by reference herein. In some embodiments, the strain sensor 202 may receive instructions from an external device, e.g., a reader, via the transceiver 205 to sense the strain in a medical device such as a bone plate, a spinal plate, or a spinal rod.

After sensing the strain, the strain sensor 202 may store the sensed strain in the non-volatile memory 204 as a strain value among other previously stored strain values 206. In other embodiments, the strain sensor 202 may receive instructions from an external device via the transceiver 205 to sense the strain in a medical device and transmit a strain value to the external device via the transceiver 205. In embodiments, the transceiver 205 may transmit strain values 206 stored in non-volatile memory 204 to an external device.

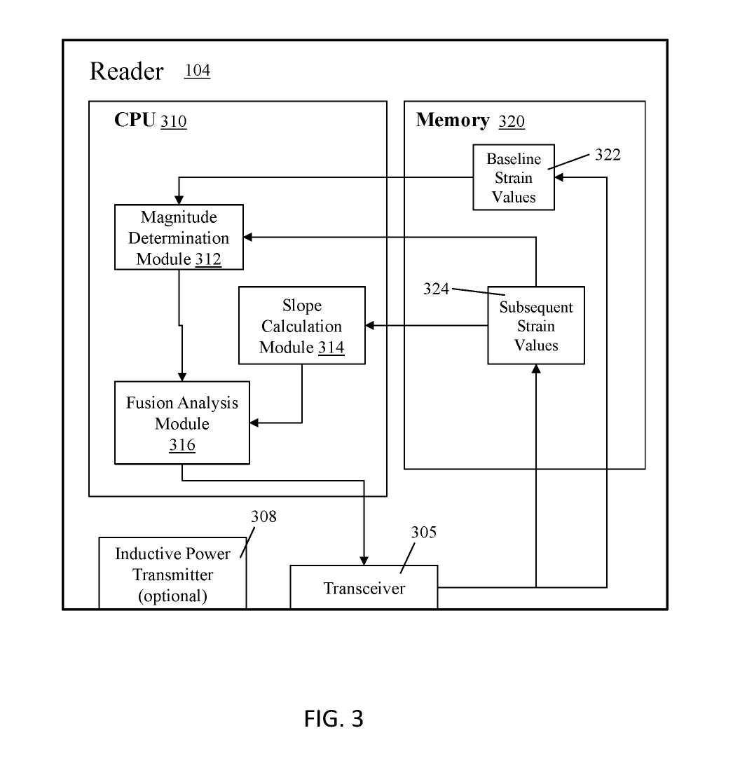

FIG. 3 is a block diagram illustrating a reader 104 of the system architecture 100 of FIG. 1. The reader includes a transceiver 305, an optional inductive power transmitter, a central processing unit (CPU) 310, and a memory 320. In embodiments, CPU 310 can execute various applications which include instruction sets stored in memory 320. In some embodiments, the reader 104 may include a display device, e.g., an LCD or one or more LEDs (indicating whether bone fusion is successful), and/or a user interface (not shown). The CPU 310 may be implemented in a digital signal processor (DSP), an application specific integrated circuit (ASIC), or a field programmable gate array (FPGA).

The memory 320 can be any device, physical structure, and/or populated data structure which functions as a recorded media storage device. In some embodiments, memory 320 may include computer memory which is volatile, that is, memory which does not maintain its state when an electric current is no longer available. Memory 320 may include non-volatile memory, dynamic memory, and/or redundant memory. Examples of such memory include random access memory (RAM), optical memory devices, magnetic media, disc hard drives, solid state hard drives, SDRAM, DDR RAM, erasable programmable read-only memories (EEPROMs), or other media for storing data for future retrieval or modification. In accordance with some embodiments, memory 320 can be contained within one contiguous region of a physical device, may span across multiple regions on a physical device, or may span multiple memory devices such as virtual memory allocated within non-volatile memory space.

In embodiments, the inductive power transmitter 308 is an inductive coil. The reader 104 may include a power source (not shown) coupled to the inductive coil, which supplies a current through the inductive coil, which may, in turn, induce a current in an inductive coil of a sensor system, e.g., the inductive coil in the inductive power receiver 208 of the sensor system 200 of FIG. 2.

In embodiments, the transceiver 305 receives strain values and stores them in memory 320 as baseline strain values 322 and subsequent strain values 324. The CPU 310 executes a magnitude determination module 312, which accesses the memory 320 to obtain baseline strain values 322 and the subsequent strain values 324 and calculates the magnitude difference between the obtained baseline strain values 322 and the subsequent strain values 324. The CPU 310 also executes a slope calculation module 314, which accesses the memory 320 to obtain subsequent strain values 324. The slope calculation module 314 calculates a slope value based on multiple subsequent strain values 324, e.g., three consecutive, subsequent strain values 324. The CPU 310 also executes a fusion analysis module 316, which analyses the results of the magnitude determination module 312 and the slope calculation module 314 and determines the status or progress of the bone fusion. For example, the fusion analysis module 316 may determine that the bone fusion is complete or is not complete. In embodiments, the fusion analysis module 316 may generate a message indicating the status or progress of the bone fusion and the transceiver 305 may transmit the message to an external device, e.g., the computer 108, which may display the message on display 110.

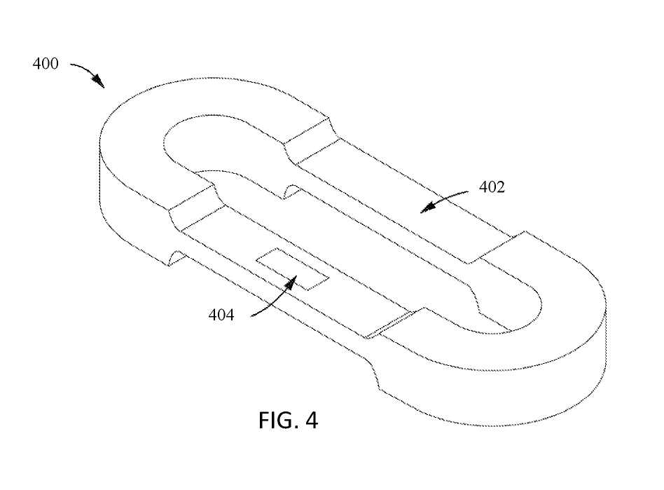

FIG. 4 is a perspective view of a spinal plate 400 on which a sensor system 404 is disposed in accordance with some embodiments. As shown in FIG. 4, the sensor system 404 is an electromechanical device which is affixed to a surface of a machined portion 402 of the spinal plate 400 as described in commonly owned U.S. Pat. No. 8,721,570, which is incorporated by reference herein. In other embodiments, the sensor system 404 is embedded within the machined portion 402 of the spinal plate 400. In embodiments, the sensor system may also be embedded in a spinal rod or bone plate. Examples of bone plates in which the sensor system 404 may be embedded include the bone plates disclosed in commonly-owned U.S. Pat. Nos. 8,303,633; 8,403,969; 8,574,272; and 8,636,738, the contents of each of which are incorporated by reference herein.

Since the spinal rod or spinal plate is implanted in the patient permanently, the long term safety of the sensor system may be needed. In embodiments, the sensor system may be housed in a biocompatible, hermetically-sealed housing and can remain in the body indefinitely. In embodiments, the strain sensor system does not include a battery or active electronics, but includes inductive coils that receive power from an external inductive power source and transmits strain measurement data wirelessly to an external reader device. When interrogated by the external reader device, the strain sensor system may power on, measure the current level of strain, and transmit this information wirelessly to the external reader device. In embodiments, the electronic circuitry of the strain sensor system may capture a short period of data, convert it to digital information, average hundreds to thousands of data points into one or several data points, and transmit the one or several data points to the external reader device for further processing by software running on the external reader device.

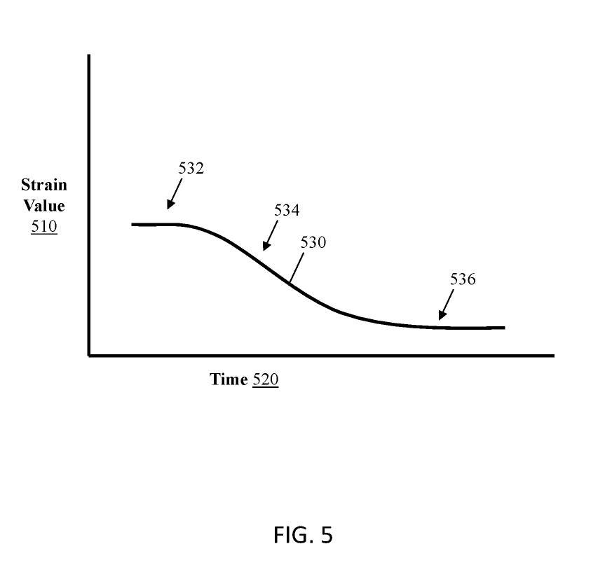

FIG. 5 is a graphical diagram illustrating a strain curve 530 of a spinal device over time 520 after a spinal fusion procedure in accordance with embodiments of the present disclosure. After a spinal procedure, the slope at the beginning portion 532 of the strain curve 530 is at or near zero. Then, over the course of several weeks or months, the bony structures advance through a fusing process and gain more strength, and thus take on more of the load that was placed on the spinal device at the time it was deployed in the spine. This is illustrated by the middle portion 534 of the strain curve 530, where the slope is a negative value. In some embodiments, the initial strain value in the spinal plate or rod is reversed, which results in a slope of a positive value and decreasing magnitude. As the spinal fusion nears completion, the strain values make little to no change. This is illustrated by the latter portion 536 of the strain curve 530, where the slope of the strain curve 530 approaches a zero value. The fusion may take over two years to finalize, so the slope of the strain curve 530 is at a near-zero value, which is determined through empirical testing, etc.

In embodiments, the methods of the present disclosure determine whether bone fusion, e.g., spinal fusion, is sufficiently developed to carry the mechanical load by determining that the measured strain values reach the latter portion 536 of the strain curve 530.

According to embodiments of the present disclosure, the average strain is taken from the most recent two or more strain data points and the slope is calculated. In embodiments, this slope is compared to the prior slope and the change in slope is determined. Typically, the slope will initially be flat but the absolute value of strain will have a high initial value, as illustrated by the beginning portion 532 of the strain curve 530. As the cartilaginous fusion begins to form, the slope will become steeper as illustrated by the middle portion 534 of the strain curve 530, showing a developing fusion. After several weeks, this slope will begin to decrease as the fusion reaches a mechanical strength sufficient to carry the load as illustrated by the latter portion 536 of the strain curve 530. Once the slope has decreased below a threshold, which is determined empirically, the spine is considered fused. The spinal fusion process continues for an additional two years, but after some number of weeks, based on the strain slope, it achieves enough mechanical strength for the patient to safely resume normal activities.

It is important to rule out other conditions that would cause a relatively flat slope, such as an initial state before the fusion fracture healing response begins as illustrated by the beginning portion 532 of the strain curve 530, or in the incidences of delayed union, non-union, or pseudo-union, where the cartilaginous mass does not stiffen and mineralize into bone. Thus, the second criterion of the disclosed method is whether there is a step change in magnitude of strain. Again, this will be based on empirical data collection. In embodiments, the method of the present disclosure concludes that fusion has occurred if a threshold for change in magnitude of the strain from the baseline is exceeded and a flat slope is present. The surgeon may have access to the generated strain curve for their patient, but they do not need to consult it in order to know where their patient is in the fusion process. The basic statistics may be available to the surgeon from the external reader, which indicates how much the strain has changed and what the slope is.

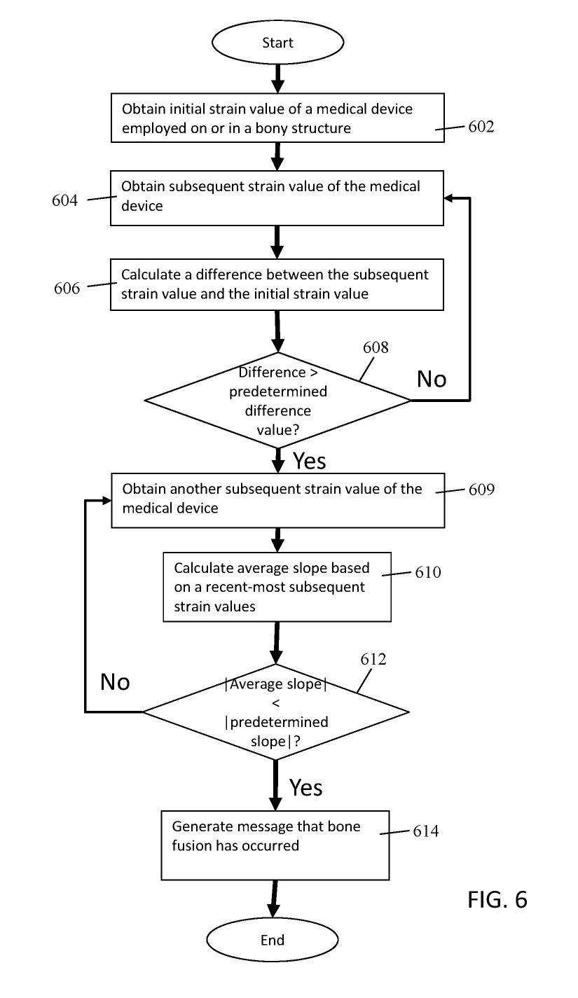

FIG. 6 is a flow diagram illustrating an example process for monitoring the status of bone fusion in accordance with some embodiments. After starting, an initial strain value of a medical device disposed on or in a bony structure is obtained at block 602. For example, an external reader device may obtain the initial strain value by accessing a memory device incorporated into a strain sensor system disposed on or in the medical device. The medical device may be any suitable medical device including spinal plate 400 with sensor system 404 (as illustrated in FIG. 4). At block 604, a subsequent strain value of the medical device is obtained after an interval, e.g., a week or other appropriate period. The patient may be given instructions to regularly visit a clinic where a clinician uses the external reader device to obtain the subsequent strain value. Alternatively, a sensor device system having an energy storage device may regularly measure strain and store corresponding strain values in non-volatile memory of the strain sensor system so that multiple strain values can be obtained using the external reader device at a single visit to the clinic. This would reduce the number of visits to the clinic by the patient.

The intervals at which strain measurements are taken depend on the embodiment, e.g., whether the strain measurements are taken via a wearable reader. In some embodiments, I the strain measurements may be taken once a month. In other embodiments, a clinician may use a “6 week, 3 month, 6 month, 1 year” time interval to take strain measurements as the clinician may not see any change in the bony structure via X-ray until 6 months after surgery.

At block 606 a difference between the subsequent strain value and the initial strain value is calculated. At block 608, it is determined whether the difference between the subsequent strain value and the initial strain value is greater than a predetermined difference value. The predetermined difference value is a value at which there is a high probability that the fusion process is properly progressing. The predetermined difference value may be determined based on strain values obtained from patients who used the same or similar medical device and/or who have a medical condition that is the same or similar as the medical condition of the patient. If the difference between the subsequent strain value and the initial strain value is not greater than the predetermined difference value, the process returns to block 604 to obtain another subsequent strain value of the medical device.

If the difference between the subsequent strain value and the initial strain value is greater than the predetermined difference value, another subsequent strain value of the medical device is obtained at block 609 and the average slope is calculated based on recent-most subsequent strain values, e.g., the recent-most two or three subsequent strain values, at block 610. At block 612, it is determined whether the average slope is less than a predetermined slope. If the average slope is less than the predetermined slope, a message or notification that bone fusion has occurred is generated at block 614. The message or notification may be a text message (e.g., “Fusion is Complete”) presented on a display (e.g., a liquid crystal display (LCD) display) of a reader device. Alternatively, the message or notification may simply involve illuminating a single light emitting diode (LED) incorporated into the reader device. If the average slope is not less than the predetermined slope, the process returns to block 609 to obtain another subsequent strain value of the medical device.

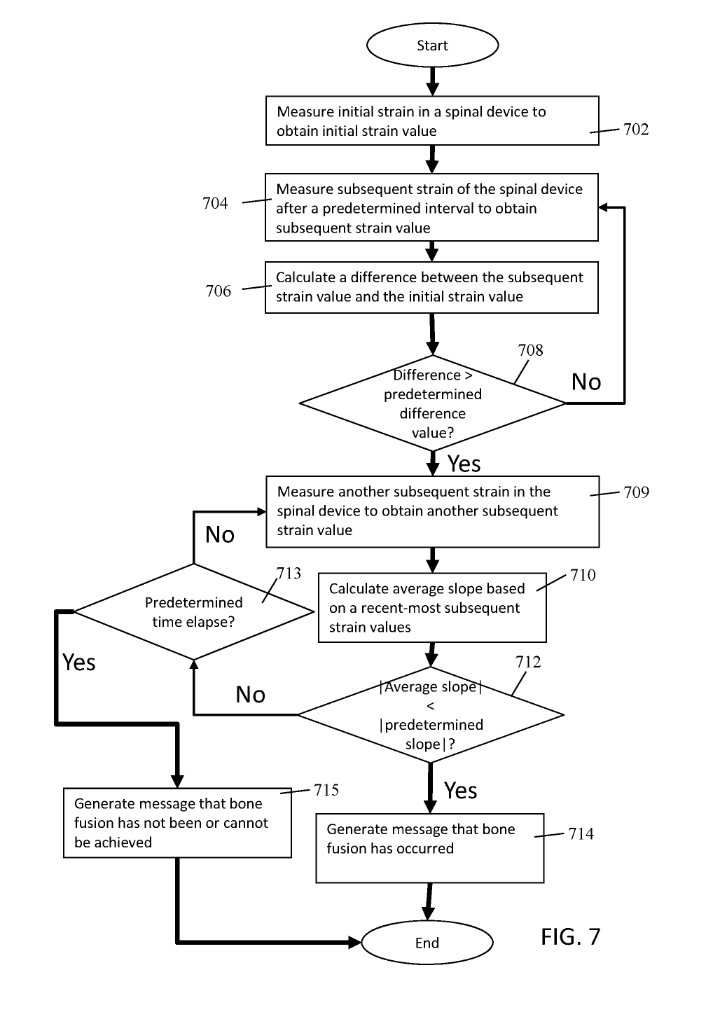

FIG. 7 is a flow diagram illustrating an example process for monitoring the status of spinal fusion in accordance with some embodiments. In embodiments, the example process of FIG. 7 may be performed by a strain sensor system disposed on or in a spinal device, such as a spinal plate or spinal rod. After starting, an initial or baseline strain in a spinal device implanted in or disposed on a spinal bony structure is measured to obtain an initial or baseline strain value at block 702. The spinal device may be any suitable spinal device including spinal plate 400 with sensor system 404 (as illustrated in FIG. 4). At block 704, a subsequent strain in the spinal device is measured after a predetermined interval to obtain a subsequent strain value. At block 706, a difference between the subsequent strain value and the initial strain value is calculated.

At block 708, it is determined whether the difference between the subsequent strain value and the initial strain value is greater than a predetermined difference value. If the difference is not greater than the predetermined difference value, the process returns to block 704 to measure subsequent strain in the spinal device to obtain another subsequent strain value. If the difference is greater than the predetermined difference value, another subsequent strain in the spinal device is measured to obtain another subsequent strain value at block 709 and the average slope is calculated based on recent-most subsequent strain values at block 710. At block 712, it is determined whether the average slope is less than a predetermined slope. If the average slope is less than the predetermined slope, a message that spinal fusion has occurred is generated at block 714; otherwise, it is determined whether a predetermined time has elapsed at block 713.

If it is determined that the predetermined time has not elapsed at block 713, the process returns to block 709, at which another subsequent strain in the spinal device is measured to obtain another subsequent strain value. If it is determined that the predetermined time has elapsed at block 713, a message that spinal fusion has not been or cannot be achieved is generated at block 715. The message may indicate that spinal fusion revision needs to be performed. The predetermined time may be a period, which, if exceeded, indicates a high likelihood that bone fusion will not occur or cannot be achieved.

In embodiments, the method of FIG. 7 may further include continually calculating the slope and analyzing the calculated slopes and the measure strain values to determine particular abnormalities that require spinal fusion revision surgery. Through testing, it may be determined that certain combinations of calculated slopes and measured strain values indicate particular abnormalities such as when a pedicle screw breaks, a spinal rod becomes loose, the endplate loading on the vertebrae adjacent to the fusion changes, or the bony structures develop a cartilaginous pseudo-union that does not convert into bone. For example, if the calculated slopes do not change and the measured strain values are abnormally low, the strain sensor may no longer be loaded properly. As a result, it may be determined that the spinal rod is loose or a screw is broken and a message may be generated indicating that the spinal rod is loose or a screw is broken.

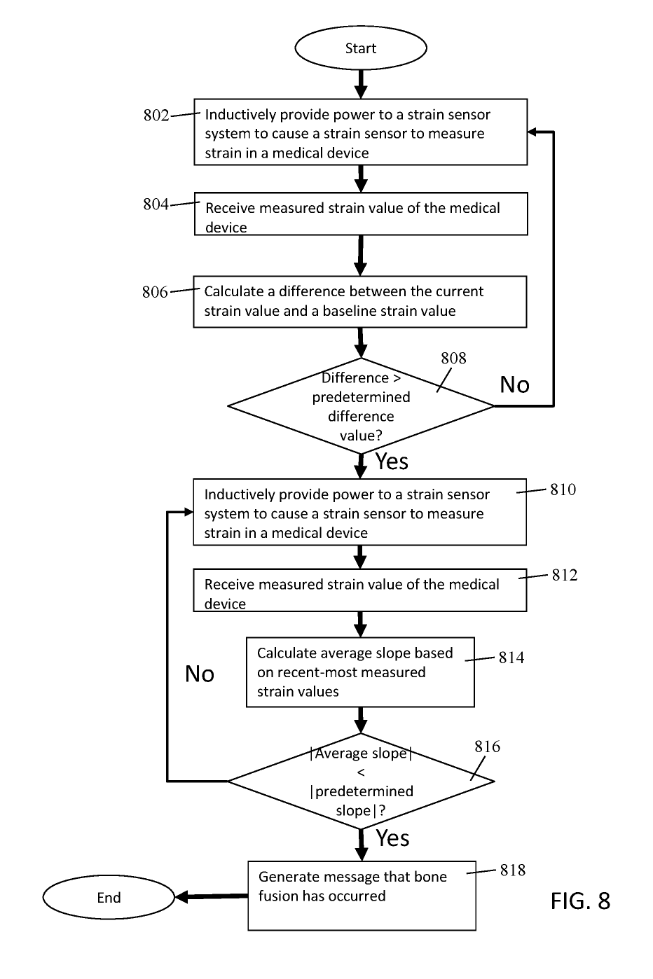

FIG. 8 is a flow diagram illustrating an example process performed by an external reader device for monitoring the status of bone fusion in accordance with some embodiments. After starting, power is inductively provided to a strain sensor system disposed on or in a medical device to cause a strain sensor to measure a strain value of the medical device at block 802. At block 804, a measured strain value of the medical device is received. The medical device may be any suitable medical device including spinal plate 400 with sensor system 404 (as illustrated in FIG. 4). At block 806, a difference between a current strain value and a baseline strain value is calculated. At block 808, it is determined whether the difference between the current strain value and the baseline strain value is greater than a predetermined difference value. If the difference between the current strain value and the baseline strain value is not greater than the predetermined difference value, the process returns to block 802 and, in embodiments, performs the function of 802 after a predetermined interval.

If the difference between the current strain value and the baseline strain value is greater than the predetermined difference value, power is inductively provided to the strain sensor system to cause the strain sensor to measure a strain value of the medical device at block 810 and the measured strain value of the medical device is received at block 812. At block 814, the average slope is calculated based on recent-most measured strain values, e.g., the recent-most two or three measured strain values. At block 816, it is determined whether the average slope is less than a predetermined slope. If the average slope is less than the predetermined slope, a message that bone fusion has occurred is generated at block 818. Otherwise, the process returns to block 810 and, in embodiments, performs the function of 810 after a predetermined interval.

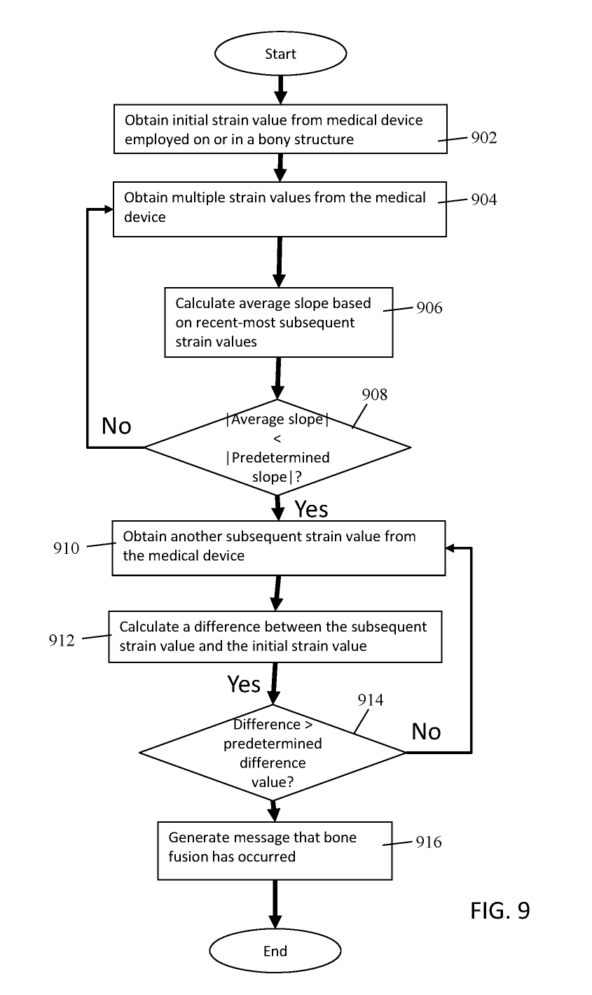

FIG. 9 is a flow diagram illustrating an example process for monitoring the status of bone fusion in accordance with other embodiments. After starting, an initial strain value of a medical device disposed on or in a bony structure is obtained at block 902. For example, an external reader device may obtain the initial strain value by accessing a memory device incorporated into a strain sensor system disposed on or in the medical device. The medical device may be any suitable medical device including spinal plate 400 with sensor system 404 (as illustrated in FIG. 4). At block 904, multiple strain values are obtained from the medical device after an interval, e.g., a week or other appropriate period. At block 906, the average slope is calculated based on recent-most subsequent strain values, e.g., the recent-most two or three subsequent strain values. At block 908, it is determined whether the average slope is less than a predetermined slope.

If the absolute value or magnitude of the average slope is less than the absolute value or magnitude of a predetermined slope, a subsequent strain value of the medical device is obtained after an interval, e.g., a week or other appropriate period, at block 910. The patient may be given instructions to regularly visit a clinic where a clinician uses the external reader device to obtain the subsequent strain value. Alternatively, a sensor device system having an energy storage device may regularly measure strain and store corresponding strain values in non-volatile memory of the strain sensor system so that multiple strain values can be obtained using the external reader device at a single visit to the clinic. This would reduce the number of visits to the clinic by the patient. If the absolute value or magnitude of the average slope is not less than the predetermined slope, the process returns to block 609 to obtain another subsequent strain value of the medical device.

At block 912, a difference between the subsequent strain value and the initial strain value is calculated. At block 914, it is determined whether the difference between the subsequent strain value and the initial strain value is greater than a predetermined difference value. The predetermined difference value is a value at which there is a high probability that the fusion process is properly progressing. In other embodiments, the predetermined difference value may be an indication that the fusion is strong enough to bear the mechanical load, which means that the patient may resume normal activities. The predetermined difference value may be determined based on strain values obtained from patients who used the same or similar medical device and/or who have a medical condition that is the same or similar as the medical condition of the patient. If the difference between the subsequent strain value and the initial strain value is not greater than the predetermined difference value, the process returns to block 910 to obtain another subsequent strain value of the medical device.

If the difference between the subsequent strain value and the initial strain value is greater than the predetermined difference value, a message or notification that bone fusion has occurred is generated at block 916. The message or notification may be a message including text (e.g., “Fusion is Complete”) presented on a display (e.g., a liquid crystal display (LCD) display) of a reader device. Alternatively, the message or notification may simply involve illuminating one or more light emitting diodes (LEDs) incorporated into the reader device.

While several embodiments of the disclosure have been shown in the drawings, it is not intended that the disclosure be limited thereto, as it is intended that the disclosure be as broad in scope as the art will allow and that the specification be read likewise. Any combination of the above embodiments is also envisioned and is within the scope of the appended claims. Therefore, the above description should not be construed as limiting, but merely as exemplifications of particular embodiments. Those skilled in the art will envision other modifications within the scope and spirit of the claims appended hereto. For example, while the present disclosure makes reference to the spine, the present disclosure contemplates application of the systems and methods to other types of bony structures, such as bony structures in an arm, hand, leg, or foot.

In another embodiment, a spinal fusion monitoring process may include continually measuring strain and calculating a slope based on the measured strain. The process may further include determining whether the magnitude of the slope is greater than a predetermined slope value, and determining that bone fusion has started progressing at a desired rate, if it is determined that the magnitude of the slope is greater than the predetermined slope value. The process may further include (1) determining whether the magnitude of the slope is greater than the predetermined slope for a predetermined time, and (2) determining whether the magnitude of slope is less than a second predetermined value. If (1) and (2) are determined to be true, then the process may generate a message or a notification that spinal fusion has occurred successfully.

In embodiments, strain measurements and/or slope calculations may be used to determine when and/or how much an implanted growing rod should be lengthened or shortened. In some embodiments, strain measurements and/or slope calculations may be used to control a magnetically-controlled growing rod (MCGR). The reader may take strain measurements and/or make slope calculations, and, based on those measurements and/or calculations, calculate how much the rod should be lengthened or shortened. Information including the amount by which the rod should be lengthened or shortened may then be wirelessly transmitted or otherwise provided to a remote controller, which causes the rod to be lengthened or shortened via, for example, a telescopic actuator portion of the rod.

In embodiments, subsequent strain measurements taken during the fusion process may be used to predict the progress and status of the bone fusion.

In embodiments, the spinal rod loads may be assessed during surgery using the strain sensors. The strain sensors could be used as a “not to exceed” tool for strain, which could prevent breaking spinal rods, including long scoliosis rods, during surgery.

1. A method, comprising:
measuring an initial strain in a medical device implanted in a bony structure to obtain an initial strain value;
measuring a subsequent strain in the medical device to obtain a subsequent strain value;
calculating a difference between the subsequent strain value and the initial strain value;
determining whether a magnitude of the difference is greater than a predetermined difference value; and
in response to determining that the magnitude of the difference is greater than the predetermined difference value,
measuring a plurality of strain values, calculating an average slope based on the plurality of measured strain values;
determining whether the average slope is less than a predetermined slope; and
in response to determining that the average slope is less than the predetermined slope, generating a notification that bone fusion in a bony structure has occurred.

2. The method of claim 1, further comprising in response to determining that the average slope is greater than the predetermined slope, generating a notification that bone fusion in a bony structure has not occurred.

3. The method of claim 1, further comprising:
determining whether a predetermined period has elapsed; and
in response to determining that the magnitude of the difference is less than the predetermined difference value, the average slope is greater than the predetermined slope, and the predetermined period has elapsed, generating a notification that a non-union or pseudo-union in the bony structure has occurred.

4. The method of claim 1, further comprising transmitting a wireless signal including the initial strain value and the subsequent strain value from a strain sensor disposed in the medical device to a reader device.

5. The method of claim 1, wherein each of the plurality of strain values are measured at a predetermined interval.

6. The method of claim 5, wherein the predetermined interval is one day, one week, or one month.

7. The method of claim 1, further comprising measuring strain at a plurality of positions on the medical device at a plurality of different times to obtain a plurality of strain values;
calculating a plurality of differences between a plurality of initial strain values and a plurality of subsequent strain values; and
determining whether the magnitude of each of the plurality of differences is greater than a respective plurality of predetermined difference values.

8. The method of claim 1, wherein the medical device is a spinal device.

9. The method of claim 8, wherein the spinal device is a spinal rod or a spinal plate.

10. The method of claim 1, further comprising:
transmitting a wireless interrogation signal from a wireless reader to a strain sensor embedded in the medical device; and
in response to receiving the wireless interrogation signal at the strain sensor, powering on the strain sensor, measuring a current strain in the medical device, and transmitting strain information based on the measured strain from the strain sensor to the wireless reader.

11. The method of claim 10, wherein the strain sensor is powered by the wireless interrogation signal.

12. The method of claim 10, further comprising transmitting the strain information from the wireless reader to a computing device; and
performing the calculating and determining steps by the computing device.

13. The method of claim 1, wherein the bony structures are spinal structures, foot structures, leg structures, or arm structures.

14. The method of claim 1, wherein the plurality of strain values include the subsequent strain value.

15. A system for performing spinal fusion, comprising:
a spinal device including a strain sensor configured to periodically measure strain in the spinal device implanted in the spine; and
a computing device in communication with the spinal device and configured to:
acquire an initial strain value from the spinal device;
acquire a subsequent strain value from the spinal device;
calculate a difference between the subsequent strain and the initial strain;
determine whether a magnitude of the difference is greater than a predetermined difference value;
if it is determined that the magnitude of the difference is greater than the predetermined difference value, measuring a plurality of strain values, calculating an average slope based on the plurality of measured strain values, and determining whether the average slope is less than a predetermined slope; and
if the average slope is determined to be less than the predetermined slope, determining that spinal fusion has been achieved.

16. The system of claim 15, wherein the computing device is further configured to:
cause the strain sensor to measure an initial strain in the spinal device; and
cause the strain sensor to measure a subsequent strain in the spinal device.

17. The system of claim 15, wherein the computing device is a reader device including a first wireless transceiver that wirelessly acquires strain values from a second wireless transceiver disposed in the spinal device and coupled to the strain sensor.

18. The system of claim 15, wherein the spinal device is a screw, a spinal rod, a cervical plate, a spinal plate, a spinal implant, an artificial disc, or a growing rod.

19. The system of claim 15, wherein the plurality of strain values are measured at a predetermined interval.

20. The system of claim 19, wherein the predetermined interval is one day, one week, or one month.

21. A system for performing spinal fusion, comprising:
a spinal device including a strain sensor configured to measure strain in the spinal device implanted in the spine; and
a computing device configured to:
acquire an initial strain value;
acquire a plurality of strain values from the spinal device;
calculate an average slope based on the plurality of acquired strain values;
determine whether an absolute value of the average slope is less than a predetermined slope;
if the absolute value of the average slope is determined to be less than the predetermined slope, acquire a subsequent strain value from the spinal device, calculate a difference between the subsequent strain value and the initial strain value, and determine whether the absolute value of the difference is greater than a predetermined difference value; and
if it is determined that the absolute value of the difference is greater than the predetermined difference value, determine that spinal fusion has been achieved.

22. The system of claim 21, wherein the spinal device further includes a communications device configured to communicate with a cloud computing system and configured to transmit and store strain values in memory or a database of the cloud computing system.

23. The system of claim 22, wherein the computing device acquires strain values from the cloud computing system.

24. The system of claim 21, wherein the computing device is at least a portion of a cloud computing system.

25. The system of claim 21, wherein the computing device is a portable electronic device, a smartphone executing an application, or a personal computer executing a software application, and
wherein the computing device is configured to acquire strain values from the strain sensor of the spinal device.

26. The system of claim 21, wherein the computing device acquires strain values from the strain sensor of the spinal device by transmitting an electromagnetic signal to the strain sensor to cause the strain sensor to sense and transmit a strain value to the computing device.

27. The system of claim 21, wherein the computing device acquires strain values from the strain sensor of the spinal device by generating an electrical field and inducing a current in a coil of the strain sensor to cause the strain sensor to sense and transmit an electromagnetic signal including strain value information to the computing device.

28. The system of claim 21, wherein the spinal device further includes memory for storing a plurality of strain values, and
wherein the computing devices acquires the plurality of strain values from the memory when the computing device comes in close proximity to the spinal device.
